(19) United States
(12) Patent Application Publication
(10) Pub. No.: US 2003/0170230 A1
(43) Pub. Date: Sep. 11, 2003

(54) Compositions and methods for assembly and stabilization of antibody Fv fragments via antiparallel heterogeneous coiled-coil peptide regions and uses thereof

(76) Inventors: Nigel Robert Caterer, Copenhagen S (DK); Lars Otto Uttenthal, Copenhagen East (DK); Rasmus Wendelbo Nielsen, Copenhagen N (DK)

Correspondence Address: LICATLA & TYRRELL P.C., 66 E. MAIN STREET, MARLTON, NJ, 08053, US

(21) Appl. No.: 10/360,053

(22) Filed: Feb. 5, 2003

Related U.S. Application Data
(60) Provisional application No. 60/354,376, filed on Feb. 5, 2002.

(51) Int. Cl. A61K 39/395; C07K 16/00

(52) U.S. Cl. 424/130.1; 530/387.1

(57) ABSTRACT

Compositions and methods using antiparallel heterogeneous α-helical coiled-coil (AHEC) regions for the linkage and stabilization of antibody Fv domains are provided.

This application claims the benefit of priority from U.S. Provisional Application Serial No. 60/354,376, filed Feb. 5, 2002, which is hereby incorporated by reference in its entirety.

### FIELD OF THE INVENTION

The present invention relates to the use of antiparallel heterogeneous dimeric, trimeric and tetrameric coiled-coil (AHEC) peptide regions, specifically designed de novo, selected from libraries, or derived from nature, for the assembly and stabilization of antibody Fv fragments in a predetermined manner. Use of the AHEC region permits the assembly of antibody fragments into defined multimeric complexes, wherein the essential feature of the AHEC is the stabilization of two antibody Fv domains and their possible linkage to a different functional group or polypeptide chain, including a further pair of antibody Fv domains. The two antibody Fv domains are expressed as N-terminal fusion proteins in which the C-terminal fusion partner is one of the AHEC complex peptides. When assembled, the AHEC complex holds the C-terminals of the two Fv domains at each end of the AHEC complex. The distance between the two AHEC fusion ends is optimized to be similar to that found in full-length antibodies at around 35-45 Å. The use of AHEC peptides that form trimeric or tetrameric complexes allows the addition of further functional proteins, protein fragments, peptides or chemically modified peptides to the AHEC-antibody Fv complex. Multimeric complexes joined via AHEC regions are useful in a number of different areas including, but not limited to, research, industry and healthcare.

### BACKGROUND OF THE INVENTION

Various coiled-coil multimerization regions for the assembly of proteins or protein fragments have been described.

WO 98/56906 describes tetranectin-derived polypeptides capable of forming stable trimers. These complexes comprise the tetranectin trimerization region as the trimerizing structural element for other protein and chemical entities. WO 95/31540 describes a trimerization module derived from collectin coiled-coil structures and its application to the engineering of artificially trimerized proteins. Polypeptides comprising a collectin neck region that are able to trimerize are also described in U.S. Pat. No. 6,190,886.

Coiled-coil multimerization regions have also been used in various contexts in relation to the production and use of recombinant antibodies. U.S. Pat. No. 5,643,731 describes uses for a pair of leucine-zipper peptides, preferably v-fos and c-jun, for in vitro diagnosis, in particular for the immunochemical detection and determination of an analyte in a biological fluid. In one method, the first leucine-zipper peptide is immobilized by attaching it to a solid support, the second leucine-zipper peptide is coupled to a specific binding partner for the analyte, and the amount of analyte bound to the binding partner is determined. U.S. Pat. No. 6,165,335 describes a biosensor apparatus for detecting a binding event between a ligand and its receptor. The apparatus includes a biosensor surface and surface-bound two-subunit heterodimer complexes composed of preferably oppositely charged peptides that together form an α-helical coiled-coil. The first peptide is attached to the biosensor surface, and the second peptide carries the ligand, accessible for binding by a ligand-binding agent. Binding of the ligand-binding agent to the surface-bound ligand is then detected in a suitable manner. A ligand-specific biosensor surface can readily be prepared from a universal template containing the first charged peptide, by addition of a selected ligand attached to the second peptide.

U.S. Pat. No. 5,932,448 describes methods for producing and using bispecific antibodies formed by leucine zippers. U.S. Pat. No. 5,837,242 describes polypeptides consisting of a first domain comprising a binding region of an immunoglobulin heavy-chain variable region, and a second domain comprising a binding region of an immunoglobulin light-chain variable region, the domains being linked but incapable of associating with each other to form an antigen binding site. These polypeptides are associated to form antigen-binding multimers, such as dimers, which may be multivalent or have multispecificity. The domains may be linked by a short peptide linker or may be joined directly together. Bispecific dimers may have longer linkers. Methods of preparation of polypeptides and multimers and diverse repertoires thereof, and their display on the surface of bacteriophage for easy selection of interest, are described.

The use of parallel helix-stabilized antibody fragments is also disclosed by Arndt et al. (J. Mol. Biol. 2001 312:221-228). The production of recombinant single chain antibody Fv fragments has also become well established since its inception over 10 years ago (Bird et al. Science 1988 242:423-426; Huston et al. Proc. Natl Acad. Sci. USA 1988 85:5879-5883).

A trimeric AHEC region can be derived from the repeated domains of spectrin. Spectrin, also referred to as fodrin, is a common component of cytoskeletal structures associated with cell membranes in metazoan organisms (Shenk, M. A. and Steele, R. E. Trends Biochem Sci. 1993 18:459-463). Electron microscopic studies of spectrin have revealed a flexible elongated molecule composed of two loosely intertwined antiparallel strands that appear to be tightly associated at both ends (Shotton et al. J. Mol. Biol. 1979 131:303-329). Each of these strands contains two homologous alpha and beta chains that associate into tetramers through a head-to-head interaction. The elongated protein chains of the spectrin family contain tandemly repeated segments, each segment doubling back on itself into a S-shape containing three interacting α-helical regions. The crystal structure of the repetitive segment of spectrin is taught by Yan et al. (Science 1993 262:2027-2030). The three-dimensional structure in solution of a chicken-brain spectrin repeat determined by NMR spectroscopy and distance geometry-simulated annealing calculations is taught by Pascual et al. (J. Mol. Biol. 1997 273:740-751).

The use of spectrin as a joining component of two or more effector molecules is described in U.S. Pat. No. 5,997,861. U.S. Pat. No. 6,303,317 teaches the use of coiled-coil region peptides such as the coiled-coil region of spectrin as probes to identify target polypeptides.

AHEC complexes may also be designed de novo. The ability to select for dimeric, trimeric or tetrameric complexes has been taught in previous publications (Zhou et al. Biochemistry 1993 32:3178-3187; Harbury et al. Science 1993 262:1401-1407; Monera et al. Protein Eng. 1996 9:353-363). Selecting between parallel or anti-parallel dimeric coiled-coil formation has been taught in a number of articles (Myszka, D. G. and Chaiken, I. M. Biochemistry 1994 33:2363-2372; Monera et al. Biochemistry 1994 33:3862-3871; Monera et al. J. Biol. Chem.1993 268:19218-19227; Oakley, M. G. and Kim, P. S. Biochemistry 1998 37:12603-12610; Betz et al. Biochemistry 1997 36:2450-2458; Monera et al. J. Biol. Chem. 1996 271:3995-4001; McClain et al. J. Am. Chem. Soc. 2001 123:3151-3152). Selection of heterogeneous coiled-coil complexes has also been examined (Nautiyal et al. Biochemistry 1995 34:11645-11651; McClain et al. J. Am. Chem. Soc. 2001 123:3151-3152). The effect of cysteine position on interchain disulfide linkage has been taught for two-stranded α-helical coiled coils (Zhou et al. Biochemistry 1993 32:3178-3187).

### SUMMARY OF THE INVENTION

An object of the present invention is to provide compositions and methods for the assembly of a pair of Fv antibody fragments alone or with a protein, protein fragment, peptide or chemical in a defined manner by attaching a specific AHEC peptide to each component to be assembled. The attached antibody Fv fragments alone or with a protein, protein fragment, peptide or chemical associate into antiparallel heterogeneous dimeric, trimeric or tetrameric coiled coils, thus assembling the components into a non-naturally occurring oligomer.

Another object of the present invention is to provide compositions and methods wherein one or more cysteine residues are placed within or near the AHEC region to form interchain disulfide bridges, covalently linking two of the AHEC peptide chains as well as their attached proteins, protein fragments, peptides or chemicals, and thus stabilizing the complex, once formed by non-covalent interaction, by covalent crosslinkage.

Another object of the present invention is to provide compositions and methods using these AHEC regions to covalently or non-covalently attach proteins, protein fragments, peptides or chemical complexes to a surface and or solid support via an AHEC region.

### DESCRIPTION OF THE FIGURES

FIG. 1 provides several nonlimiting examples of non-naturally occurring multimeric proteins derived from the present invention, particularly those derived from antibody Fv fragments, where the AHEC region is used to attach other functional units. FIG. 1(a) shows how AHEC complexes may be used in phage display of Fv antibody fragments; FIG. 1(b) shows the possibility of associating a His-tag enabling the complex to bind to nickel chelating columns; FIG. 1(c) shows how an AHEC region can be used to attach inert molecules such as poly(ethylene glycol) (PEG); and FIG. 1(d) shows specific immobilization to a surface via a linking molecule containing a peptide capable of forming part of an AHEC region.

FIG. 2(a) is an overview of the structure of a single-chain left-handed antiparallel triple-helical coiled-coil spectrin repeat domain. The figure is derived from the structure determined by Pascual et al. (J. Mol. Biol. 1997 273:740-751). FIG. 2(b) shows a subdivision of a spectrin repeat into the three separate chains (A, B and C) that make up an AHEC complex.

FIG. 3 shows the expression vector pG31018 derived from a pET20b(+) expression vector (Novagene). The vector contains DNA encoding the light-chain Fv domain of the anti-tetanus toxoid antibody HYB 278-14, followed by an AHECa region and finally by an affinity tag consisting of six histidine residues. The DNA sequence is shown in lower case (SEQ ID NO:1) and the derived amino-acid sequence in the upper case single letter code (SEQ ID NO:2). Relevant regions are marked in bold with an explanation in italics above.

FIG. 4 shows the expression vector pG31020 derived from pG31018 in which the codon for valine 122 has been mutated to one for cysteine (underlined). The DNA sequence is shown in lower case (SEQ ID NO:3) and the derived amino-acid sequence in the upper case single letter code (SEQ ID NO:4). Relevant regions are marked in bold with an explanation in italics above.

FIG. 5 shows the expression vector pG31025 derived from a pET20b(+) expression vector (Novagene) and containing DNA encoding a pe1B leader peptide followed by a factor Xa cleavage site, the heavy-chain Fv domain of the anti-tetanus toxoid antibody HYB 278-14, an AHECb region, and finally an affinity tag consisting of six histidine residues. The DNA sequence is shown in lower case (SEQ ID NO:5) and the derived amino-acid sequence in the upper case single letter code (SEQ ID NO:6). Relevant regions are marked in bold with an explanation in italics above.

FIG. 6 shows the expression vector pG31030 derived from pG31025 in which the codon for valine 176 has been mutated to one for cysteine (underlined). The DNA sequence is shown in lower case (SEQ ID NO:7) and the derived amino-acid sequence in upper case single letter code (SEQ ID NO:8). Relevant regions are marked in bold with an explanation in italics above.

FIG. 7 shows the expression vector pG31010 derived from the pET20b(+) expression vector (Novagen) and containing DNA encoding a pe1B leader peptide followed by a ubiquitin domain, factor Xa cleavage site, AHECc region and finally an affinity tag consisting of six histidine residues. A single ubiquitin domain-encoding sequence was selected by PCR from a pUC19 vector containing a sequence encoding eight ubiquitin domains (Genebank entry M26880). The DNA sequences encoding the factor Xa cleavage site and AHECc region were produced by PCR using two overlapping synthetic oligonucleotides. The DNA sequence is shown in lower case (SEQ ID NO:9) and the derived amino-acid sequence in upper case single letter code (SEQ ID NO:10). Relevant regions are marked in bold with an explanation in italics above.

FIG. 8 shows the expression vector pG31027 derived from pG31010 in which the codon for arginine 107 of pG31010 has been mutated to one for serine, thus destroying the factor Xa cleavage site. Another factor Xa cleavage site (GSGIEGRM) has then been inserted in between the codons for methionine 23 and aspartic acid 24. The DNA sequence is shown in lower case (SEQ ID NO:11) and the derived amino-acid sequence in upper case single letter code (SEQ ID NO:12). Relevant regions are marked in bold with an explanation in italics above.

FIG. 9 shows the direct binding of samples to immobilized tetanus toxoid. FIG. 9(a) shows results of ELISA analysis of samples A (protein from vectors pG31018, pG31025 and pG3010) B (protein from vectors pG31020 and pG31030) and C (protein from vectors pG31020, pG31030 and pG31027). FIG. 9b shows protein concentration required for each sample to give an optical density of 1.5 in direct ELISA.

FIG. 10 shows the inhibition of antibody binding to immobilized tetanus toxoid with free tetanus toxoid. FIG. 10(a) shows results of ELISA analysis of samples A, B and C as defined in FIG. 9. FIG. 10(b) shows binding affinity of these samples and the wild type antibody.

### DETAILED DESCRIPTION OF THE INVENTION

The present invention relates to the use of peptides that form left-handed antiparallel α-helical coiled-coil complexes for the assembly and stabilization of antibody Fv fragment domains for their use as functional ligand-binding molecules. These peptides are referred to herein as AHEC peptides or AHEC peptide regions. Trimeric and tetrameric AHEC peptide regions can be used to stabilize and assemble proteins, protein fragments, peptides and/or other chemicals with the antibody Fv fragment and form multimeric complexes.

In one embodiment, dimeric AHEC peptide regions are used to stabilize pairs of Fv antibody fragment chains, holding the two chains together and approximately in the correct position. Antibodies are composed of two pairs of heavy and light chains. The heavy and light chains are folded into a number of domains that interact with each other giving the antibody its general form. About 100 amino-acid residues at the N-terminus of each chain vary greatly between different antibodies and form the variable or Fv domain. The Fv domains of both chains normally bind to each other to form the complementarity-determining region (CDR). Because of the variability of the two Fv domains, their binding affinity can be weak. The union and correct positioning of the two Fv domains are normally stabilized by the other antibody domains.

Because of their complex nature, requiring correct folding and disulfide linkage, functional antibodies are not easily produced recombinantly, making the expression systems required expensive and/or difficult to handle. This has to some degree been overcome by producing antibody fragments such as Fab fragments. Fab fragments contain the variable domain as well as the first constant domain of both the light and heavy chains, the constant domains being included to help stabilize the light and heavy chain complex. Fab fragments are, however, still relatively difficult to produce recombinantly. Another strategy to facilitate the production and use of recombinant antibody fragments is to express them as a single chain (scFv), where both the heavy and light chain variable regions are linked by a long linking peptide (e.g. (GGSG)₃). Linking the two Fv domains keeps them in close proximity to each other while they are dissociated. This format is often the easiest to produce recombinantly as it contains the minimum number of domains and disulfide linkages. However, it has been found that the length of linking peptide required varies from antibody to antibody. The N-termini of the Fv chains are also usually located close to if not within the CDR region and the addition or removal of the linking peptide has sometimes been found to affect binding affinity of the antibody. Other formats for stabilizing antibody fragments have also been investigated, including mutating each chain by inserting cysteine residues. These residues are then used to form a disulfide linkage between the two chains. However, because of the variability of the Fv regions, the mutation sites must be optimized for each antibody. The use of parallel helices to stabilize the antibody Fv regions helps overcome the problems of variations in the Fv domains; however, this requires the use of linking chains to span the distance between the two Fv C-termini. The unstructured nature of the linkage regions increases their susceptibility to proteolysis.

In Fab fragments or full-length antibodies the C-terminal ends of the two Fv domains are located in the order of 30-50 Å from each other.

Using AHEC peptide regions of the present invention, the antibody Fv domains can be placed at either end of the AHEC peptide region. Once bound, the AHEC peptide regions serve to stabilize the antibody Fv complex by holding the two chains in approximately the correct relative position without the need for a long linking peptide. In this embodiment, the antibody Fv domains are linked via AHEC peptides attached to their C-termini, and not the N-termini which participate in the CDR. Accordingly, their binding properties are less likely to be affected. This allows antibody Fv regions, derived from e.g. mouse IgG, to be used without incurring the risk of conformational changes in the antibody Fv complex due to the presence of an scFv linking peptide. As the Fv complex is stabilized in a manner similar to that in Fab fragments and full-length antibodies, the chance of successfully changing antibody formats is much higher. The Fv containing complex can be further stabilized by placing cysteine residues within or adjacent to the relevant AHEC peptide, thus permitting covalent linkage by the formation of an interchain disulfide bridge. The formation of interchain disulfide linkages has been demonstrated for two-stranded α-helical coiled-coils (Zhou et al. Biochemistry 1993 32:3178-3187).

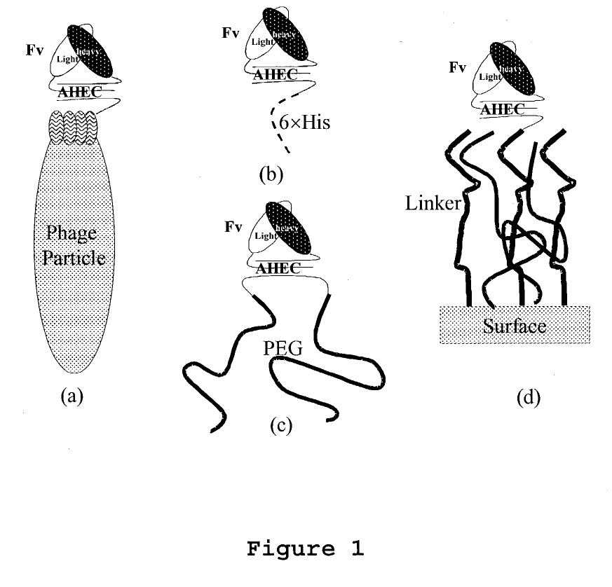

As only two AHEC chains are used for Fv stabilization, the remaining peptide or peptides in trimeric and tetrameric AHEC peptide complexes can be used for the attachment of proteins, protein fragments, peptides and/ or chemicals such as functional moieties including, but not limited to, other antibodies, affinity tags, enzymatic labels, dyes, poly(ethylene glycol) (PEG), toxins and the immobilization of the AHEC complex to a solid surface (see FIG. 1). The general advantage of the invention is that it exploits the specific binding of antibodies, but instead of retaining the Fc region, with its often undesirable function of provoking inflammation and complement activation, it provides for the ready attachment of a large number of different functional groups that can be chosen to fulfill a variety of therapeutic and diagnostic applications.

The other AHEC peptide(s) in trimeric and tetrameric AHEC peptide regions can also be used to enable the Fv-AHEC complex to be displayed of on the surface of phage particles. The use of AHEC-stabilized Fv fragments in phage display allows for the selection of Fv antibody fragments that are easily produced in Escherichia coli. Such fragments can then be used, if required, in the production of full-length antibodies.

When the two AHEC chains stabilizing the Fv complex are covalently linked to each other, the other chain(s) of the AHEC complex can be readily exchanged by dissociating the AHEC complex with an agent such as 2-8 M urea and reassociating the complex in the presence of new AHEC peptide(s) linked to the new functional groups.

The formation of multimeric proteins protected by inert molecules such as PEG permits the production of modular chimeric proteins with a broad spectrum of functions and reduced immunogenicity. The AHEC region can be used to link selected proteins or protein fragments with many varied functions. For example, in one embodiment, two immunoglobulin Fv fragments can be linked to a toxin for targeted cell killing. Alternatively, an immunoglobulin fragment can be linked to an enzyme for color reactions. Both of these exemplary multimeric proteins can be produced without having to go back to the DNA level and produce new expression vectors and then express and refold the multimeric protein. The attachment of inert molecules such as PEG to the Fv-AHEC complex reduces its immunogenicity for use in therapy. Attachment of such molecules to the AHEC region is less likely to directly cause conformational changes in the Fv complex, as may happen when they are attached directly or very close to the Fv complex, as is required in scFv. Attachment of inert molecules also reduces the amount of protein exposed to proteolytic cleavage. These two factors and the increase in the size of the complex are expected to prolong the residency time of the oligomeric protein complex in the body. A decrease in the immunogenicity of the multimeric protein is an advantage when multiple treatments are required.

Accordingly, the multimeric complexes of the present invention are useful in the production of therapeutic antibodies and/or antibody fragments. The antibodies may be used for a number of functions, including the inhibition of receptor binding and the targeting of drugs, toxins and labels. The fusion or attachment of peptides constituting a trimeric AHEC region is useful in the production of humanized mouse antibodies. Further, because of the simple nature of the modified Fv complex it can be easily expressed in E. coli, thus reducing production costs. The peptides of the AHEC region can then be used for site-specific PEGylation, protecting this region both from cleavage and from recognition by the host immune system.

Peptides capable of forming an AHEC region can also be used to attach proteins to a surface. One peptide of an AHEC region can be immobilized to a surface such as a solid support directly or via a linking molecule such as PEG. This allows either covalent or non-covalent attachment of proteins to a surface without chemical treatment. This again has the advantage in that the protein is immobilized in a specific manner and is not inactivated by non-specific adsorption or by coupling reactions. Covalent or non-covalent attachment of proteins, protein fragments, peptides or chemical complexes to a surface and or solid support via an AHEC region can be performed routinely in accordance with well known procedures. Examples of surfaces or solid supports to which the complexes of the present invention may be immobilized include, but are in no way limited to, microtiter plates, slides, culture dishes and beads.

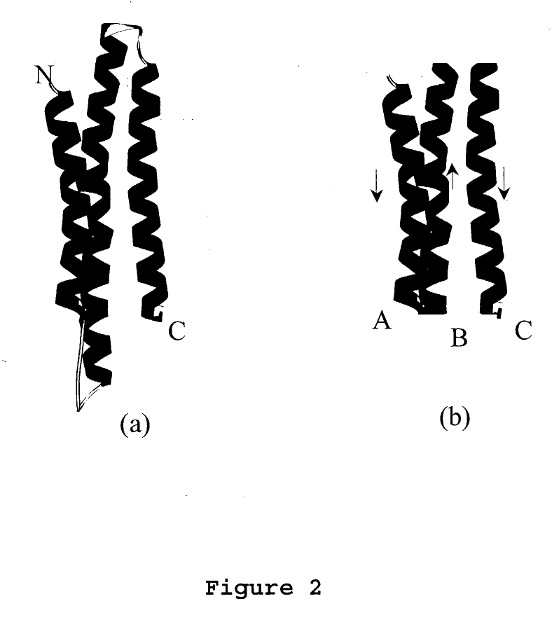

In a preferred embodiment of the present invention, peptides forming the AHEC regions are specifically designed or derived from a spectrin protein. Use of AHEC regions specifically designed or derived from a spectrin protein can improve the development of multimeric proteins for both therapeutic and diagnostic purposes. In one embodiment, non-naturally occurring multimeric proteins of the present invention are prepared using each of three α-helical coils derived from the spectrin family of proteins as separate chains (See FIG. 2b). Exemplary amino-acid sequences of the three α-helical coils derived from known spectrin repeats, namely the 16^{th} repeat of chicken brain α-spectrin (coil A is SEQ ID NO:11; coil B is SEQ ID NO:12; coil C is SEQ ID NO:13; Pascual et al. J. Mol. Biol. 1997 273:740-751) and the 14^{th} repeat of Drosophila α-spectrin (coil A is SEQ ID NO:14; coil B is SEQ ID NO:15; coil C is SEQ ID NO:16; Yan et al. Science 1993 262:2027-2030) are depicted in Table 1.

TABLE 1

Amino-acid sequences of exemplary AHEC peptides derived from spectrin

| Coil | 16^{th} repeat of chicken brain α-spectrin | 14^{th} repeat of Drosophila α-spectrin |
|---|---|---|
| A | QFFRDDEESWKKLLVSSED | RLQQLFRDVEDEETWIREKEPIAASTNRGK |
| | (SEQ ID NO:11) | (SEQ ID NO:14) |
| B | KHKRLELAAHEPAIQGVLDTG | LIKKHEDFDKAINGHEQKIAALQTVADQL |
| | (SEQ ID NO:12) | (SEQ ID NO:15) |
| C | IQQRLAQFVDHWKELKQLAARG | ASNLVDEKRKQVLERWRHLKEGLIEKRSRLG |
| | (SEQ ID NO:13) | (SEQ ID NO:16) |

In another embodiment, non-naturally occurring AHEC peptides of the present invention are prepared by de novo design. The design of AHEC peptides can also be based on the prior art for the formation of antiparallel (McClain et al. J. Am. Chem. Soc. 2001 123:3151-3152; Monera et al. J. Biol. Chem. 1993 268:19218-19227; Monera et al. Biochemistry 1994 33:3862-3871; Monera et al. J. Biol. Chem. 1996 271:3995-4001; Myszka, D. G. and Chaiken, I. M. Biochemistry 1994 33:2363-2372; Oakley, M. G. and Kim, P. S. Biochemistry 1998 37:12603-12610), dimeric (McClain et al. J. Am. Chem. Soc. 2001 123:3151-3152; Monera et al. J. Biol. Chem. 1993 268:19218-19227; Monera et al. Biochemistry 1994 33:3862-3871; Monera et al. J. Biol. Chem. 1996 271:3995-4001; Myszka, D. G. and Chaiken, I. M. Biochemistry 1994 33:2363-2372) and tetrameric (Betz et al. Biochemistry 1997 36:2450-2458; Harbury et al. Science 1993 262:1401-1407; Monera Protein Eng. 1996 9:353-363) coiled coils, examples of each being given in Table 2.

TABLE 2

Amino-acid sequences of exemplary de novo designed AHEC peptides

| Coil | Dimeric | Trimeric | tetrameric |
|---|---|---|---|
| A | QALEKELAQNEWELQALEKELAQLEKELQA | AIEYEQAAIKEEIAAIKDKIAAIKEYIA | SAQRLLKIARRLRKEAKELLKRAEHG |
| | (SEQ ID NO:17) | (SEQ ID NO:19) | (SEQ ID NO:22) |
| B | QALKKKLLAQLKWKLQALKKKNAQLKKKLQA | AILYKIAAIEEKIAQIEEEIAAQEEKIA | GPELLKKVEELEKKVDKLYKIVEHG |
| | (SEQ ID NO:18) | (SEQ ID NO:20) | (SEQ ID NO:23) |
| C | | AIKYKQAAIKNEIAAIKQEIAAIEQMIA | SAQELLKIARRLRKEAKELLKEAEHG |
| | | (SEQ ID NQ:21) | (SEQ ID NO:24) |
| D | | | GPRLLKEVEELEKKVDELYKIVEHG |
| | | | (SEQ ID NO:25) |

The following non-limiting examples are provided to further illustrate the present invention.

### EXAMPLES

### Example 1

### AHEC Peptides and Fv Fragments

AHEC peptides were selected form human spectrin (Genebank entry U83867; SEQ ID NO:26), AHECa consisting of residues 783-811, AHECb residues 825-853, and AHECc residues 858-885. The Fv sequences are derived from the mouse monoclonal anti-tetanus toxoid antibody HYB 278-14.

### Example 2

### Expression Vector Construction

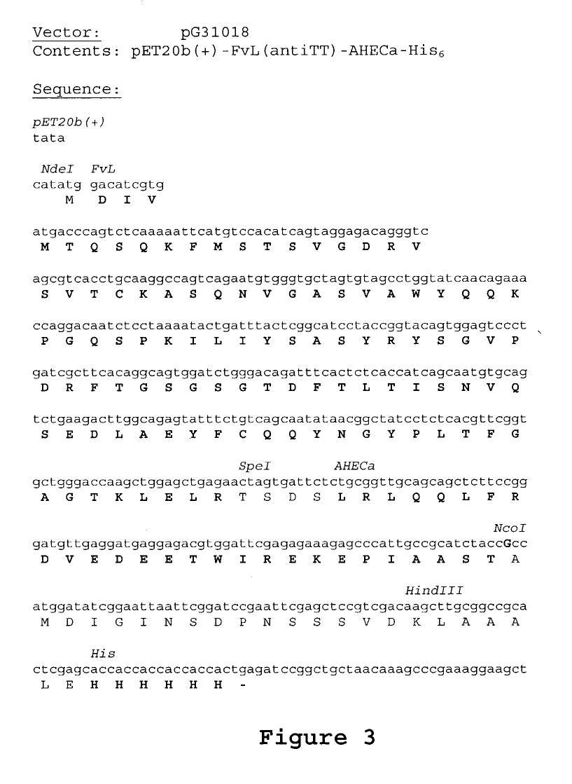

The pG31018 expression vector (FIG. 3) was derived from a pET20b(+) expression vector (Novagene) and contained DNA encoding the light-chain Fv domain of antibody HYB 278-14 followed by an AHECa region and finally by an affinity tag of six histidine residues.

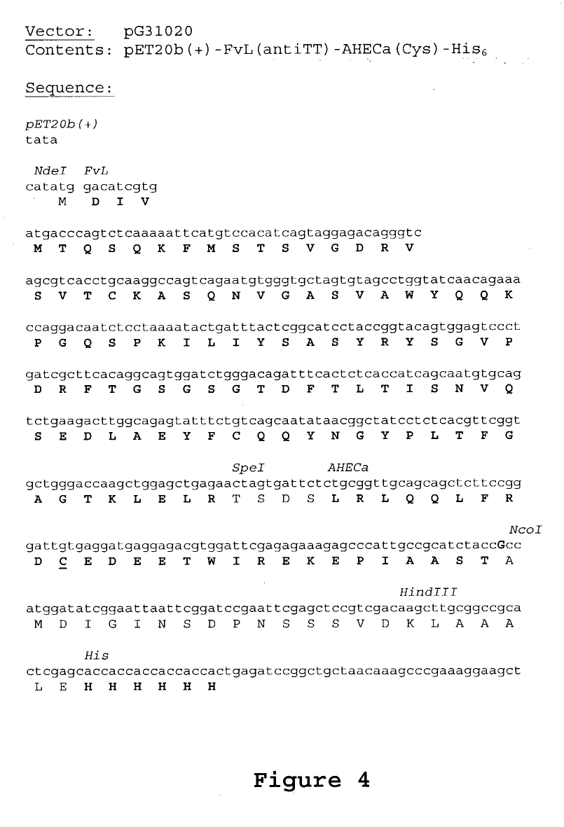

The pG31020 expression vector (FIG. 4) was derived from pG31018 by mutating the codon for valine 122 to one for cysteine.

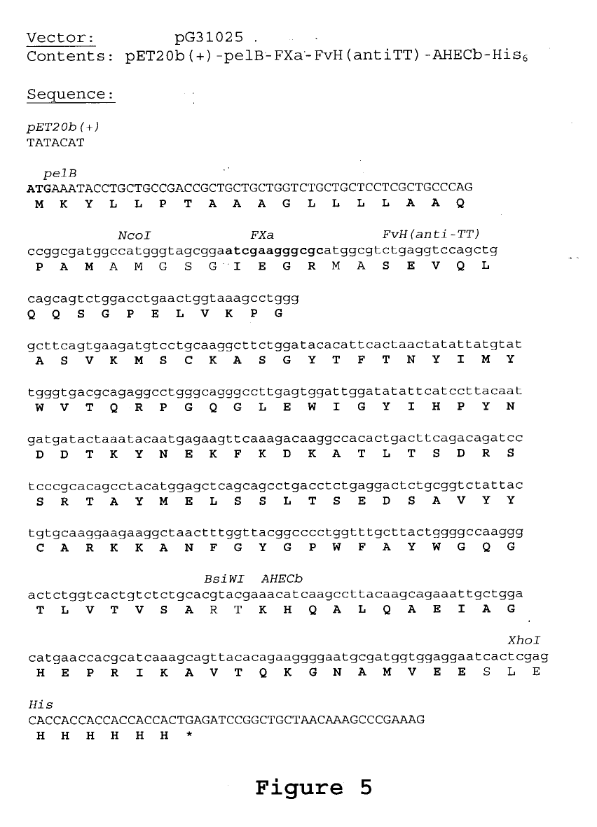

The pG31025 expression vector (FIG. 5) was derived from a pET20b(+) expression vector (Novagene) and contained DNA encoding a pe1B leader peptide followed by a factor Xa cleavage site, the heavy-chain Fv domain of antibody HYB 278-14, an AHECb region and finally an affinity tag of six histidine residues.

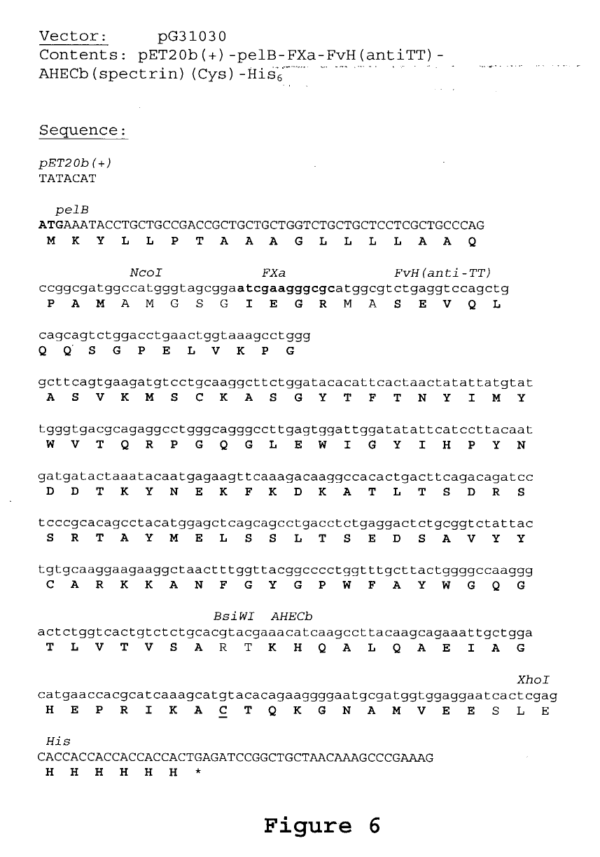

The pG31030 expression vector (FIG. 6) was derived from pG31025 by mutating the codon for valine 176 to one for cysteine.

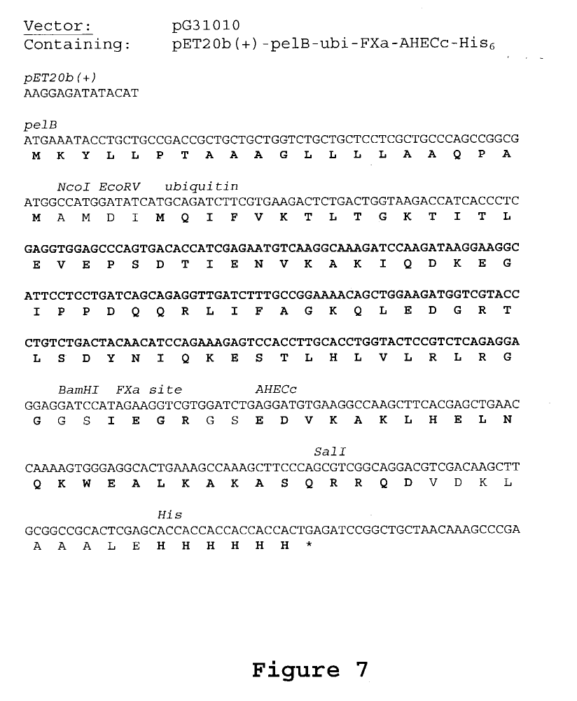

The pG31010 expression vector (FIG. 7) was derived from the pET20b(+) expression vector (Novagen) and consisted of DNA encoding a N-terminal pe1B leader peptide followed by a ubiquitin domain, factor Xa cleavage site, an AHECc region, and finally an affinity tag of six histidine residues. A single ubiquitin domain encoding sequence was selected by means of PCR from a pUC19 vector containing a sequence encoding eight ubiquitin domains (Genebank entry M26880). The DNA sequences encoding the factor Xa cleavage site and the AHECc region were produced by PCR using two overlapping synthetic oligonucleotides.

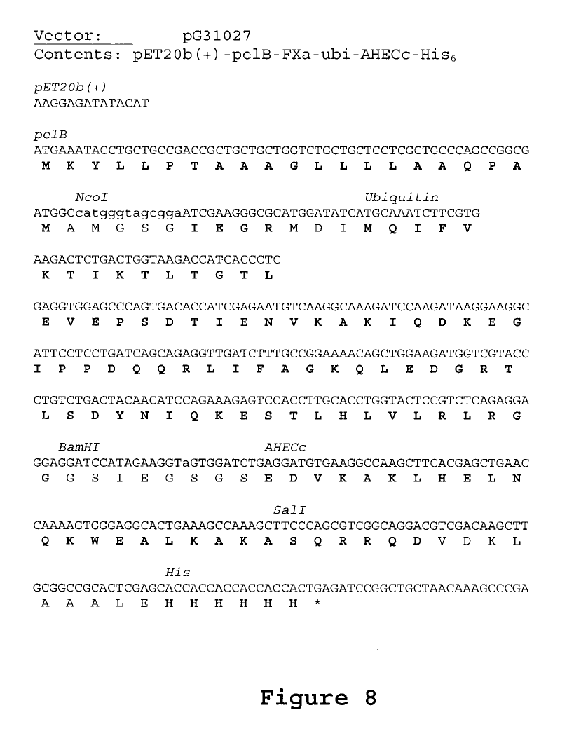

The pG31027 expression vector (FIG. 8) was derived from pG31010. Arginine 107 of pG31010 was mutated to a serine, thus destroying the factor Xa cleavage site. Another factor Xa cleavage site (GSGIEGRM (SEQ ID NO:27)) was then inserted between methionine 23 and aspartic acid 24.

### Example 3

### Protein Expression

FvL-AHECa Constructs

The expression vectors pG31018 and pG31020 were transformed into BL21(DE3) (Stratagene) E. coli by means of a standard heat-shock method. Transformed cells were selected on LB agar plates containing 100 mM ampicillin. Cultures were grown overnight at 30° C., with mixing, in 25 mL LB medium containing 100 mM ampicillin. The overnight culture was then transferred to 1 liter LB medium containing 100 mM ampicillin and incubated at 37° C. with mixing until the optical density at 600 nm of the medium was about 0.6. Expression was induced by the addition of isopropyl β-D-1-thiogalactopyranoside to a final concentration of 1 mM. Induction was carried out for three hours. The cells were harvested by centrifugation at 5000 rpm for 10 minutes at 4° C. The cell pellet was resuspended on ice in 50 ml 8 M urea, containing 500 mM NaCl, 20 mM phosphate buffer and 5 mM β-mercaptoethanol, pH 7.4. The E. coli cells were lyzed by freezing and thawing followed by sonication on ice for 5×20 seconds with a 20-second pause between cycles. Particulate matter was removed by centrifugation at 15,000 g for 20 minutes at 4° C. The supernatant was then filtered through a 0.45 μm pore-size filter ready for Ni-column purification.

FvH-AHECb Constructs

The expression vectors pG31025 and pG31030 were transformed into BL21 E. coli (Stratagene) by means of a standard heat-shock method. Transformed cells were selected on LB agar plates containing 100 mM ampicillin. Cultures were grown overnight at 30° C., with mixing, in 25 mL LB medium containing 100 mM ampicillin. The overnight culture was then transferred to 1 liter LB medium containing 100 mM ampicillin and incubated at 37° C. with mixing, until the optical density at 600 nm of the medium was about 0.6. Expression was induced by the introduction of λCE6 phage to a final concentration of 4×10⁹ pfu/ml. Induction was carried out for three hours. The cells were harvested by centrifugation at 5000 rpm for 10 minutes at 4° C. The cell pellet was resuspended on ice in 50 ml 8 M urea containing 500 mM NaCl, 20 mM phosphate buffer and 5 mM β-mercaptoethanol, pH 7.4. The E. coli were lyzed by freezing and thawing followed by sonication on ice for 5×20 seconds with a 20-second pause between cycles. Particulate matter was removed by centrifugation at 15,000 g for 20 minutes at 4° C. The supernatant was then filtered through a 0.45 μm pore-size filter ready for Ni-column purification.

Ubiquitin-AHECc Constructs

The expression vectors pG31010 and pG31027 were transformed into BL21(DE3) (Stratagene) and NovoBlue(DE3) (Novagen) E. coli, respectively, by means of a standard heat-shock method. Transformed cells were selected on LB agar plates containing 100 mM ampicillin. Cultures were grown overnight at 30° C., with mixing, in 25 mL LB medium containing 100 mM ampicillin. The overnight culture was then transferred to 1 liter LB medium containing 100 mM ampicillin and incubated at 37° C. with mixing, until the optical density at 600 nm of the medium was about 0.6. Expression was induced by the addition of isopropyl β-D-1-thiogalactopyranoside to a final concentration of 1 mM. Induction was carried out for three hours. The cells were harvested by centrifugation at 5000 rpm for 10 minutes at 4° C. The cell pellet was resuspended on ice in 50 ml 8 M urea containing 500 mM NaCl, 20 mM phosphate buffer and 5 mM β-mercaptoethanol, pH 7.4. The E. coli were lyzed by freezing and thawing followed by sonication on ice for 5×20 seconds with a 20-second pause between cycles. Particulate matter was removed by centrifugation at 15,000 g for 20 minutes at 4° C. The supernatant was then filtered through a 0.45 μm pore-size filter ready for Ni-column purification.

### Example 4

### Protein Purification

The affinity tag consisting of six histidine residues was used to purify all protein constructs on a prepacked 5-ml Ni²⁺ chelating (Ni-ETA) column (Pharmacia). All liquid chromatography was carried out on an ÄKTA prime system (Pharmacia). The Ni-ETA column was first washed with 10-20 ml wash buffer (20 mM phosphate buffer, pH 7.4, containing 8 M urea, 500 mM NaCl, 20 mM EDTA and 5.0 mM β-mercaptoethanol) followed by 20 ml eluting buffer (20 mM phosphate buffer, pH 7.4, containing 8 M urea, 500 mM NaCl, 300 mM imidazole and 5.0 mM β-mercaptoethanol). The column was then loaded with 5 ml 10 mM NiCl₂ and washed with another 25 ml eluting buffer. The Ni-ETA column was then equilibrated with 20 ml loading buffer (20 mM phosphate buffer, pH 7.4, containing 8 M urea, 500 mM NaCl, 1 mM imidazole and 0.5 mM β-mercaptoethanol). The expression extract (Example 2) was then loaded onto the column at a flow rate of 2.0 mL per minute and washed with the loading buffer until a stable optical density baseline was achieved. At this point the column was eluted with an 80-ml buffer gradient to 100% eluting buffer, 8-ml fractions being collected. All the constructs emerged from the column as broad peaks with a maximum at around 66% elution buffer. Fractions containing this peak were then pooled for analysis.

The protein concentration of each construct was estimated by measuring the optical density at 280 nm. The theoretical extinction coefficient for each construct was determined from its amino-acid sequence according to Gill and von Hippel (Anal. Biochem. 1989 182:319). The calculated values are given in Table 3.

TABLE 3
Summary of parameters calculated for each
antibody fragment construct.

| Protein | Expression | Molecular weight | E_{280 nm} 0.1% |
|---|---|---|---|
| containing | vector | (Da) | (= 1 g/l) |
| FvL | pG31018 | 18614.6 | 1.099 |
| | pG31020 | 18618.6 | 1.099 |
| FvH | pG31025 | 21678.5 | 1.764 |
| | pG31030 | 21682.5 | 1.764 |
| Ubiquitin | pG31010 | 16815.3 | 0.491 |
| | pG31027 | 17534.1 | 0.471 |

### Example 5

### Protein Folding and Factor Xa Treatment

In the current example three separate combinations of the purified protein were examined: (A) Protein from vectors pG31018, pG31025 and pG31010; (B) Protein from vectors pG31020 and pG31030; (C) Protein from vectors pG31020, pG31030 and pG31027.

For each folding, equal amounts of the purified constructs were combined in a 3.5 kDa cutoff dialysis tube and placed in 250 ml buffer A (8 M urea, 500 mM NaCl, 50 mM Tris-HCl, pH 8.0, 5 mM EDTA, 2 mM glutathione). This was allowed to equilibrate for 2-4 hours before folding was commenced. 1 liter of buffer B (500 mM NaCl, 50 mM Tris-HCl, pH 8.0, 5 mM EDTA, 2/0.2 mM reduced/oxidized glutathione) was then steadily added to buffer B with mixing over 24 hours. The total buffer volume was kept at 250 ml. On completion of the process, the folding mixture was dialyzed into 150 mM NaCl, 50 mM Tris-HCl, pH 8.0. The mixture was then centrifuged at 15,000 g for 20 minutes and then filtered through a 0.80-μm pore-sized filter. An extinction coefficient averaged between each component in the folding mixture was used to estimate the protein concentration. Factor Xa was added to the samples to a mass ratio of 1:50 to the estimated protein in the sample. This was then allowed to react overnight at 4° C.

### Example 6

### Analysis by Direct Binding ELISA

The three folded protein samples (A, B and C) were analyzed for direct binding to tetanus toxoid. MaxiSorp microtiter plates (Nunc) were coated overnight at 4° C. with 100 μl/well of 2 μg/ml tetanus toxoid in phosphate-buffered saline (PBS). The plates were washed 3×3 minutes with wash buffer (10 mM phosphate buffer, pH 7.2, containing 0.5 M NaCl and 0.1% v/v Triton X-100). The samples were diluted to a total protein concentration of 256 μg/ml in dilution buffer (wash buffer containing 1.0% w/v bovine serum albumin. Four-fold serial dilutions of the samples were prepared and added to the wells at 100 μl/well. The plate was then incubated for one hour at room temperature before washing as previously described. Bound antibody fragments were detected by means of a horseradish peroxidase-labeled anti-His-tag antibody (R931-25, Invitrogen) diluted 1/4000 in dilution buffer. The plate was incubated for a further hour and then washed as previously described. The plate was then developed with substrate solution containing 0.4 mg/ml ortho-phenylenediamine (OPD) and 0.4 μl/ml 35% hydrogen peroxide in 65 mM phosphate/35 mM citrate buffer, pH 5.0.

### Example 7

### Analysis By Inhibition ELISA

Known amounts of tetanus toxoid and or diphtheria toxoid (10-0 μg/ml) in dilution buffer were then incubated with the construct samples (A and B 64 μg/ml, C 16 μg/ml) overnight at 4° C. Samples of 100 μl of the incubates were then transferred to MaxiSorp microtiter plates (Nunc) coated with tetanus toxoid as previously described. Plates were incubated for one hour, washed and bound antibody fragments were detected by means of horseradish peroxidase-labeled anti-His-tag antibody (R931-25, Invitrogen) diluted 1/4000 in dilution buffer. The plate was incubated for a further hour, washed and developed with substrate solution as described above.

### Example 8

### Binding Properties of Construct Combinations

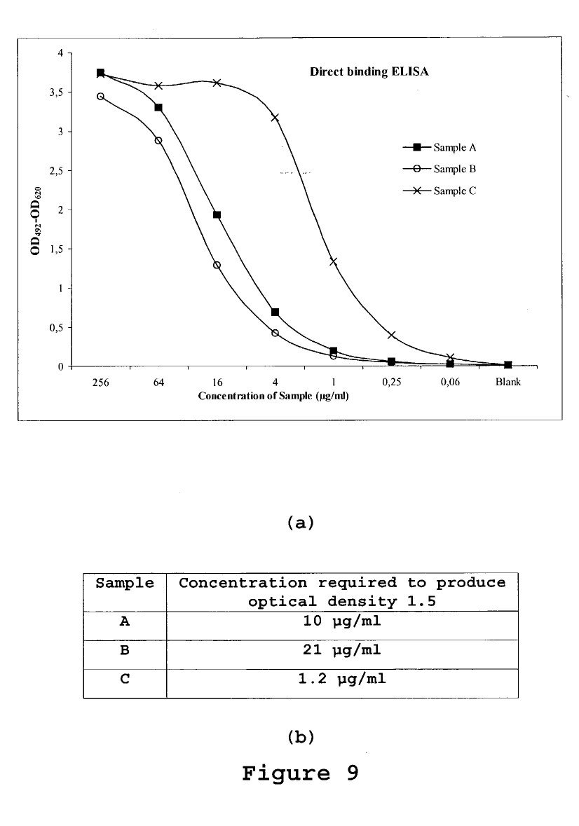

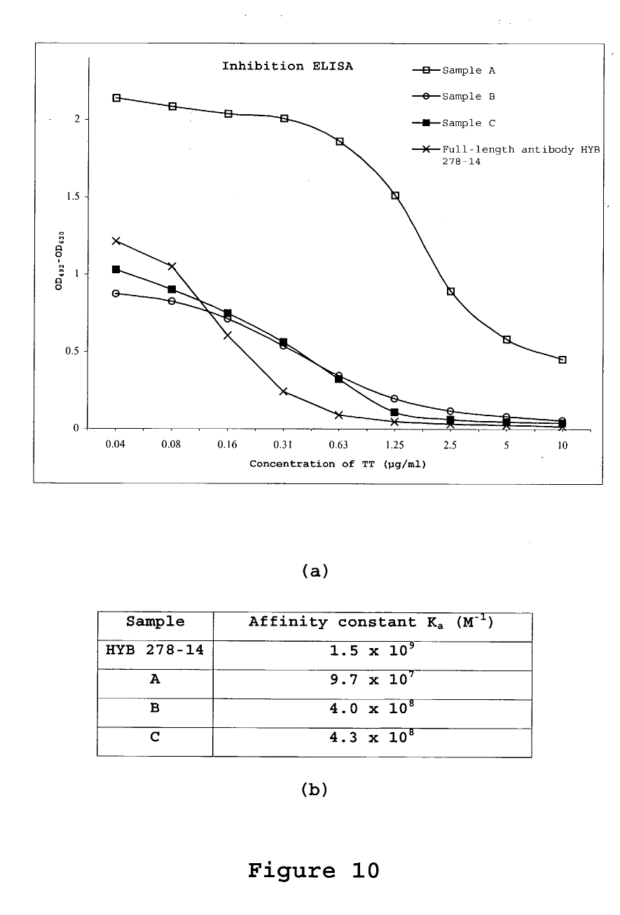

The six constructs summarized in Table 3 were expressed and purified as described. The molecular weight and purity was examined by mass spectroscopy and SDS-PAGE. Three separate combinations of the constructs were produced: A) pG31018, pG31025 and pG31010, consisting of FvL-AHECa; FvH-AHECb and AHECc without disulfide linkage; B) pG31020 and pG31030, consisting of FvL-AHECa and FvH-AHECb stabilized by a disulfide bridge; C) pG31020, pG31030 and pG31027, consisting of FvL-AHECa; FvH-AHECb and ubiquitin-AHECc with a disulfide bridge between AHECa and AHECb. The direct binding of these samples to tetanus toxoid is shown in FIG. 9. The concentrations required to give optical density values of 1.5 values (FIG. 9b) relate to both the relative concentration and affinity of functional antigen binding sites (FBS) in the samples. The ability of the construct combinations to bind specifically to tetanus toxoid was examined in an inhibition assay in which the sample was first incubated with a serial dilution of free tetanus toxoid. Then the amount of binding to immobilized tetanus toxoid was determined (FIG. 10a). This showed that all construct combinations bound specifically to tetanus toxoid. Binding of both samples B and C to the tetanus toxoid coat could be totally inhibited with free tetanus toxoid, whereas 20% of the binding of sample A could not be inhibited with free tetanus toxoid, indicating that sample A showed some non-specific interaction. Values for the affinity constants of the construct combinations and the parent antibody were determined from the inhibition assay and are shown in FIG. 10b. Samples B and C have similar affinities for tetanus toxoid, whereas sample A shows a four-fold lower affinity. This is likely to be due to the dissociation of the FvL and FvH complex disturbing the FBS. The formation of a disulfide linkage between AHECa and AHECb in samples B and C covalently attaches the FvL and FvH chains, reducing dissociation of the FBS. As stated earlier the lower affinity of the antibody construct of sample A will also affect the total concentration at which it gives an optical density of 1.5 on direct binding. Because of the lower affinity, more FBS in sample A is needed to achieve the same direct binding. Calculating the relative amounts of FBS in samples A, B and C from the data of FIGS. 9b and 10b shows that sample A contains about half as much FBS (53%) as sample C, and that sample B, lacking AHECc, contains about 6% of the amount of FBS in sample C. This shows that FBS formation occurs more readily when all three AHEC chains are present and that disulfide bridging stabilizes the FBS.

In summary, these results show that sample C, which contains all three AHEC components and is disulfide linked, is able to form more FBS with a higher affinity than the other two samples. Comparison of samples B and C also shows that once the construct combination is disulfide-stabilized, the affinity achieved is not greatly affected by the presence or absence of the third AHEC member (AHECc).

What is claimed is:

1. A composition comprising a pair of antibody Fv fragments linked and stabilized by antiparallel heterogeneous α-helical coiled-coil (AHEC) peptides.

2. The composition of claim 1 wherein the AHEC peptides form dimeric α-helical coiled-coil complexes.

3. The composition of claim 1 wherein the AHEC peptides form trimeric α-helical coiled-coil complexes.

4. The composition of claim 1 wherein the AHEC peptides form tetrameric α-helical coiled-coil complexes.

5. The composition of claim 2 wherein the AHEC peptides comprise α-helical coils specifically designed de novo.

6. The composition of claim 3 wherein the AHEC peptides comprise α-helical coils derived from repeat domains of the spectrin family proteins.

7. The composition of claim 3 wherein the AHEC peptides comprise α-helical coils specifically designed de novo.

8. The composition of claim 4 wherein the AHEC peptides comprise α-helical coils specifically designed de novo.

9. The composition of claims 3 through 4 and 6 through 8 further comprising a protein, protein fragment, peptide or chemical linked to one or more of the AHEC peptides.

10. The composition of claim 3 through 4 and 6 through 8 further comprising an inert molecule.

11. The composition of claim 9 wherein the inert molecule comprises poly(ethylene glycol).

12. An immobilized multimeric protein comprising the composition of claim 1 immobilized to a solid support via the AHEC peptides wherein one of the peptides forming the AHEC is linked to the solid support.

13. A method for stabilizing and assembling a pair of antibody Fv fragments into a multimeric complex comprising linking the pair of antibody Fv fragment via their C termini with antiparallel heterogeneous α-helical coiled-coil (AHEC) peptides.

14. The method of claim 13 wherein the AHEC peptides form trimeric α-helical coiled-coil complexes or tetrameric α-helical coiled-coil complexes.

15. The method of claim 14 further comprising linking a protein, protein fragment, peptide or chemical to the multimeric complex via one or more of the AHEC peptides.


SEQUENCE LISTING (29 sequences)

SEQ ID NO: 1 - DNA, 543 bp, Artificial sequence
  Synthetic
catatggacatcgtgatgacccagtctcaaaaattcatgtccacatcagtaggagacagggtcagcgtcacctgcaaggccagtcagaatgtgggtgctagtgtagcctggtatcaacagaaaccaggacaatctcctaaaatactgatttactcggcatcctaccggtacagtggagtccctgatcgcttcacaggcagtggatctgggacagatttcactctcaccatcagcaatgtgcagtctgaagacttggcagagtatttctgtcagcaatataacggctatcctctcacgttcggtgctgggaccaagctggagctgagaactagtgattctctgcggttgcagcagctcttccgggatgttgaggatgaggagacgtggattcgagagaaagagcccattgccgcatctaccgccatggatatcggaattaattcggatccgaattcgagctccgtcgacaagcttgcggccgcactcgagcaccaccaccaccaccactgagatccggctgctaacaaagcccgaaaggaagct

SEQ ID NO: 2 - protein, 168 aa, Artificial sequence
  Synthetic
MDIVMTQSQKFMSTSVGDRVSVTCKASQNVGASVAWYQQKPGQSPKILIYSASYRYSGVPDRFTGSGSGTDFTLTISNVQSEDLAEYFCQQYNGYPLTFGAGTKLELRTSDSLRLQQLFRDVEDEETWIREKEPIAASTAMDIGINSDPNSSSVDKLAAALEHHHHHH

SEQ ID NO: 3 - DNA, 543 bp, Artificial sequence
  Synthetic
catatggacatcgtgatgacccagtctcaaaaattcatgtccacatcagtaggagacagggtcagcgtcacctgcaaggccagtcagaatgtgggtgctagtgtagcctggtatcaacagaaaccaggacaatctcctaaaatactgatttactcggcatcctaccggtacagtggagtccctgatcgcttcacaggcagtggatctgggacagatttcactctcaccatcagcaatgtgcagtctgaagacttggcagagtatttctgtcagcaatataacggctatcctctcacgttcggtgctgggaccaagctggagctgagaactagtgattctctgcggttgcagcagctcttccgggattgtgaggatgaggagacgtggattcgagagaaagagcccattgccgcatctaccgccatggatatcggaattaattcggatccgaattcgagctccgtcgacaagcttgcggccgcactcgagcaccaccaccaccaccactgagatccggctgctaacaaagcccgaaaggaagct

SEQ ID NO: 4 - protein, 168 aa, Artificial sequence
  Synthetic
MDIVMTQSQKFMSTSVGDRVSVTCKASQNVGASVAWYQQKPGQSPKILIYSASYRYSGVPDRFTGSGSGTDFTLTISNVQSEDLAEYFCQQYNGYPLTFGAGTKLELRTSDSLRLQQLFRDCEDEETWIREKEPIAASTAMDIGINSDPNSSSVDKLAAALEHHHHHH

SEQ ID NO: 5 - DNA, 615 bp, Artificial sequence
  Synthetic
atgaaatacctgctgccgaccgctgctgctggtctgctgctcctcgctgcccagccggcgatggccatgggtagcggaatcgaagggcgcatggcgtctgaggtccagctgcagcagtctggacctgaactggtaaagcctggggcttcagtgaagatgtcctgcaaggcttctggatacacattcactaactatattatgtattgggtgacgcagaggcctgggcagggccttgagtggattggatatattcatccttacaatgatgatactaaatacaatgagaagttcaaagacaaggccacactgacttcagacagatcctcccgcacagcctacatggagctcagcagcctgacctctgaggactctgcggtctattactgtgcaaggaagaaggctaactttggttacggcccctggtttgcttactggggccaagggactctggtcactgtctctgcacgtacgaaacatcaagccttacaagcagaaattgctggacatgaaccacgcatcaaagcagttacacagaaggggaatgcgatggtggaggaatcactcgagcaccaccaccaccaccactgagatccggctgctaacaaagcccgaaag

SEQ ID NO: 6 - protein, 195 aa, Artificial sequence
  Synthetic
MKYLLPTAAAGLLLLAAQPAMAMGSGIEGRMASEVQLQQSGPELVKPGASVKMSCKASGYTFTNYIMYWVTQRPGQGLEWIGYIHPYNDDTKYNEKFKDKATLTSDRSSRTAYMELSSLTSEDSAVYYCARKKANFGYGPWFAYWGQGTLVTVSARTKHQALQAEIAGHEPRIKAVTQKGNAMVEESLEHHHHHH

SEQ ID NO: 7 - protein, 615 aa, Artificial sequence
  Synthetic
ATGAAATACCTGCTGCCGACCGCTGCTGCTGGTCTGCTGCTCCTCGCTGCCCAGCCGGCGATGGCCATGGGTAGCGGAATCGAAGGGCGCATGGCGTCTGAGGTCCAGCTGCAGCAGTCTGGACCTGAACTGGTAAAGCCTGGGGCTTCAGTGAAGATGTCCTGCAAGGCTTCTGGATACACATTCACTAACTATATTATGTATTGGGTGACGCAGAGGCCTGGGCAGGGCCTTGAGTGGATTGGATATATTCATCCTTACAATGATGATACTAAATACAATGAGAAGTTCAAAGACAAGGCCACACTGACTTCAGACAGATCCTCCCGCACAGCCTACATGGAGCTCAGCAGCCTGACCTCTGAGGACTCTGCGGTCTATTACTGTGCAAGGAAGAAGGCTAACTTTGGTTACGGCCCCTGGTTTGCTTACTGGGGCCAAGGGACTCTGGTCACTGTCTCTGCACGTACGAAACATCAAGCCTTACAAGCAGAAATTGCTGGACATGAACCACGCATCAAAGCATGTACACAGAAGGGGAATGCGATGGTGGAGGAATCACTCGAGCACCACCACCACCACCACTGAGATCCGGCTGCTAACAAAGCCCGAAAG

SEQ ID NO: 8 - protein, 195 aa, Artificial sequence
  Synthetic
MKYLLPTAAAGLLLLAAQPAMAMGSGIEGRMASEVQLQQSGPELVKPGASVKMSCKASGYTFTNYIMYWVTQRPGQGLEWIGYIHPYNDDTKYNEKFKDKATLTSDRSSRTAYMELSSLTSEDSAVYYCARKKANFGYGPWFAYWGQGTLVTVSARTKHQALQAEIAGHEPRIKACTQKGNAMVEESLEHHHHHH

SEQ ID NO: 9 - DNA, 480 bp, Artificial sequence
  Synthetic
atgaaatacctgctgccgaccgctgctgctggtctgctgctcctcgctgcccagccggcgatggccatggatatcatgcagatcttcgtgaagactctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggaggaggatccatagaaggtcgtggatctgaggatgtgaaggccaagcttcacgagctgaaccaaaagtgggaggcactgaaagccaaagcttcccagcgtcggcaggacgtcgacaagcttgcggccgcactcgagcaccaccaccaccaccactgagatccggctgctaacaaagcccga

SEQ ID NO: 10 - protein, 151 aa, Artificial sequence
  Synthetic
MKYLLPTAAAGLLLLAAQPAMAMDIMQIFVKTLTGKTITLEVEPSDTIENVKAKIQDKEGIPPDQQRLIFAGKQLEDGRTLSDYNIQKESTLHLVLRLRGGGSIEGRGSEDVKAKLHELNQKWEALKAKASQRRQDVDKLAAALEHHHHHH

SEQ ID NO: 11 - DNA, 504 bp, Artificial sequence
  Synthetic
atgaaatacctgctgccgaccgctgctgctggtctgctgctcctcgctgcccagccggcgatggccatgggtagcggaatcgaagggcgcatggatatcatgcaaatcttcgtgaagactctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggaggaggatccatagaaggtagtggatctgaggatgtgaaggccaagcttcacgagctgaaccaaaagtgggaggcactgaaagccaaagcttcccagcgtcggcaggacgtcgacaagcttgcggccgcactcgagcaccaccaccaccaccactgagatccggctgctaacaaagcccga

SEQ ID NO: 12 - protein, 159 aa, Artificial sequence
  Synthetic
MKYLLPTAAAGLLLLAAQPAMAMGSGIEGRMDIMQIFVKTIKTLTGTLEVEPSDTIENVKAKIQDKEGIPPDQQRLIFAGKQLEDGRTLSDYNIQKESTLHLVLRLRGGGSIEGSGSEDVKAKLHELNQKWEALKAKASQRRQDVDKLAAALEHHHHHH

SEQ ID NO: 13 - protein, 19 aa, Gallus sp.
QFFRDDEESWKKLLVSSED

SEQ ID NO: 14 - protein, 21 aa, Gallus sp.
KHKRLELAAHEPAIQGVLDTG

SEQ ID NO: 15 - protein, 22 aa, Gallus sp.
IQQRLAQFVDHWKELKQLAARG

SEQ ID NO: 16 - protein, 30 aa, Drosophila sp.
RLQQLFRDVEDEETWIREKEPIAASTNRGK

SEQ ID NO: 17 - protein, 29 aa, Drosophila sp.
LIKKHEDFDKAINGHEQKIAALQTVADQL

SEQ ID NO: 18 - protein, 31 aa, Drosophila sp.
ASNLVDEKRKQVLERWRHLKEGLIEKRSRLG

SEQ ID NO: 19 - protein, 30 aa, Artificial sequence
  Synthetic
QALEKELAQNEWELQALEKELAQLEKELQA

SEQ ID NO: 20 - protein, 31 aa, Artificial sequence
  Synthetic
QALKKKLLAQLKWKLQALKKKNAQLKKKLQA

SEQ ID NO: 21 - protein, 28 aa, Artificial sequence
  Synthetic
AIEYEQAAIKEEIAAIKDKIAAIKEYIA

SEQ ID NO: 22 - protein, 28 aa, Artificial sequence
  Synthetic
AILYKIAAIEEKIAQIEEEIAAQEEKIA

SEQ ID NO: 23 - protein, 28 aa, Artificial sequence
  Synthetic
AIKYKQAAIKNEIAAIKQEIAAIEQMIA

SEQ ID NO: 24 - protein, 24 aa, Artificial sequence
  Synthetic
SAQRLLKIARRLRKEAKELLKRAE

SEQ ID NO: 25 - protein, 25 aa, Artificial sequence
  Synthetic
GPELLKKVEELEKKVDKLYKIVEHG

SEQ ID NO: 26 - protein, 26 aa, Artificial sequence
  Synthetic
SAQELLKIARRLRKEAKELLKEAEHG

SEQ ID NO: 27 - protein, 25 aa, Artificial sequence
  Synthetic
GPRLLKEVEELEKKVDELYKIVEHG

SEQ ID NO: 28 - protein, 2477 aa, Homo sapien [sequence omitted]

SEQ ID NO: 29 - protein, 8 aa, Artificial sequence
  Synthetic
GSGIEGRM